SEQUENCE LISTING (8 sequences)

SEQ ID NO: 1 - DNA, 20 bp, Artificial Sequence
  Description of Artificial Sequence Primer
ccccatgcttacaagcaagt

SEQ ID NO: 2 - DNA, 20 bp, Artificial Sequence
  Description of Artificial Sequence Primer
tggctttatgtactatgtac

SEQ ID NO: 3 - DNA, 22 bp, Artificial Sequence
  Description of Artificial Sequence Primer
gaactaggtcagcccggtactt

SEQ ID NO: 4 - DNA, 22 bp, Artificial Sequence
  Description of Artificial Sequence Primer
cggagcaccaattattaacggc

SEQ ID NO: 5 - DNA, 22 bp, Artificial Sequence
  Description of Artificial Sequence Primer
ttctcaggatatacccttgaca

SEQ ID NO: 6 - DNA, 22 bp, Artificial Sequence
  Description of Artificial Sequence Primer
gaaagagcccattgaggaaatc

SEQ ID NO: 7 - DNA, 20 bp, Artificial Sequence
  Description of Artificial Sequence Primer
ccctaagcctcctaatccgt

SEQ ID NO: 8 - DNA, 20 bp, Artificial Sequence
  Description of Artificial Sequence Primer
aggaatgatggggcaagtaa

(19) United States
(12) Patent Application Publication
(10) Pub. No.: US 2004/0126796 A1
(43) Pub. Date: Jul. 1, 2004

(54) Extraction of DNA from biological samples

(76) Inventors: David Carlson, Gaithersburg, MD (US); James G. Lazar, Bethesda, MD (US); Michael Connolly, Ijamsville, MD (US)

Correspondence Address: HELLER EHRMAN WHITE & MCAULIFFE LLP, 1666 K STREET,NW, SUITE 300, WASHINGTON, DC, 20006, US

(21) Appl. No.: 10/679,378

(22) Filed: Oct. 7, 2003

Related U.S. Application Data
(60) Provisional application No. 60/416,228, filed on Oct. 7, 2002.

(51) Int. Cl. C12Q 1/68; C07H 21/04; C12N 1/08

(52) U.S. Cl. 435/6; 435/270; 536/25.4

(57) ABSTRACT

Methods and compositions are provided for extracting DNA from tissues, hair, teeth, bone, plant material, solid matrices and other samples from which DNA extraction is generally regarded as being difficult. DNA may quickly be extracted from samples, for example, in just 5 minutes to 2 hours, without enzymatic digestion or the use of toxic organic chemicals such as phenol, chloroform, guanidine thiocyanate or 2-mercaptoethanol.

This application claims priority to application Ser. No. 60/416,228, filed Oct. 7, 2002, the contents of which are hereby incorporated by reference in their entirety.

### FIELD OF THE INVENTION

The invention provides compositions, methods, and kits that are useful for purifying biomolecules, particularly DNA from biological samples.

### BACKGROUND

Mitochondrial DNA (mtDNA) is routinely used for forensic analysis and identification and is preferred for identification analysis because it is present in hundreds to thousands of copies per cell. Mitochondrial DNA is the only nucleic acid that can be recovered from extremely small or very old or degraded samples, and mitochondrial DNA extraction and analysis is most frequently performed on difficult samples such as old tissues, hair, bone and teeth. These tissues do not have sufficient nuclear DNA for analysis, but are a rich source of mitochondrial DNA

In spite of its usefulness for analysis, extraction of mitochondrial DNA from hair, bone and teeth is exceptionally difficult, and few procedures have been published. Current techniques for extracting mtDNA from hair, bone, and teeth are lengthy, laborious, and require the use of toxic organic chemicals. In addition, physical disruption such as grinding is required to release the DNA from the sample. In order to prevent contamination of other samples, the instruments used for disrupting the sample are usually discarded after a single use or must be scrupulously cleaned with concentrated acids before re-use, making the procedure expensive and time-consuming.

Commercial products for extraction of mtDNA from hair are available, but they utilize toxic organic chemicals, grinding or other physical disruption, require an enzymatic digestion, or require the presence of the hair root which contains a significant number of skin cells as well as the hair shaft itself. The commercial products are also expensive, may contain or require the use of toxic organic chemicals and are laborious. There is also a frequent need to extract and analyze mitochondrial DNA from hair shafts that do not contain a hair root or from other samples that do not contain well preserved whole cells such as teeth and bone. A well-known and widely used method for extracting mitochondrial DNA from hair is the method published by Wilson et al. This protocol has been adopted by the FBI DNA Analysis Unit II as the US forensic standard for extraction of mitochondrial DNA from hair. This protocol begins with grinding of the hair, followed by a 2-24 hour incubation with Proteinase K, extraction of the nucleic acid with phenol-chloroform-isoamyl alcohol (PCIA), and concentration of the extracted DNA in a centrifugal microconcentrator. The time required to complete the extraction of just one sample may take 2-3 days and the procedure involves the use of the highly toxic organic compounds, phenol and chloroform.

Extraction of chloroplast and mitochondrial DNA from plant material also is exceedingly difficult. Previously published methods for extraction of mitochondrial and chloroplast DNA from plants are very expensive, time consuming, (requiring very long gradient centrifugation), require the use of toxic organic chemicals, or are simply not effective in yielding sufficient DNA at a sufficiently high purity level. (Herrmann, 1982; Bookjans et al., 1984; Palmer, 1986; Maliga et al., 1995) (mtDNA: Crouzillat et al., 1987; Kohler et al., 1991; Mackenzie, 1994). Baker et al. used a modified guanidinium thiocyanate extraction procedure in combination with glass milk to extract mitochondrial DNA from hair and teeth. However, this procedure utilized toxic guanidine thiocyanate and required that the samples be finely ground.

FTA® paper from Whatman Bioscience is a specialty chemically treated paper for the preservation of nucleic acids. When biological samples such as blood are spotted onto FTA paper, the chemical treatment in the paper lyses and the DNA is immobilized on the filter paper. Amplification or restriction enzyme digestion of the DNA in the sample is performed directly on the treated paper since before this invention, there has not been a successful way to remove the DNA from the FTA paper. Biological samples stored on FTA paper are extremely stable and it is standard practice in many fields to store samples spotted on FTA paper for long periods of times and to prepare sample archives on FTA paper. Generally, only one reaction is possible from each 2 mm punch of paper that has been spotted with a biological sample since the DNA remains bound to the paper during any subsequent reaction or manipulation. Therefore, it would be highly desirable to be able to purify the DNA away from the FTA paper and recover it in a form that allows for multiple uses. Furthermore, it would be highly desirable if the DNA could be recovered through the use of a device that incorporates a DNA-binding membrane so that the recovered DNA is highly pure and is not contaminated with other biomolecules that could interfere with downstream processes such as amplification, sequencing or cloning.

### SUMMARY OF THE INVENTION

The present invention combines the use of a novel lysis buffer with a streamlined silica binding protocol to extract DNA from tissues, hair, teeth, bone, plant material, solid matrices and other samples from which DNA extraction is generally regarded as being difficult. The invention allows DNA to be quickly extracted from samples, for example, in just 5 minutes to 2 hours, depending on the type of sample, compared to the many hours or days required for methods in the prior art. In contrast to the prior art, the present invention does not require use of enzymatic digestion, or the use of toxic organic chemicals such as phenol, chloroform, guanidine thiocyanate or 2-mercaptoethanol. The present invention eliminates or dramatically reduces the need for grinding of samples prior to lysis and requires far fewer steps than the methods of the prior art and therefore enables the processing of multiple sample in parallel.

In accordance with one aspect of the invention, there is provided a method for extracting DNA from a biological sample, comprising contacting the sample with a highly basic solution comprising an effective concentration of a chelating agent, an effective concentration of a stabilizing agent and an effective concentration of a buffering agent. The chelating agent may be an alkali metal gluconate salt, for example, sodium or potassium gluconate. The stabilizing agent may be an alkali metal silicate salt, for example, sodium or potassium silicate. The buffering agent may be an alkali metal phosphate salt, for example, sodium or potassium phosphate.

The chelating agent may be present in a concentration of about 1-500 mM, for example, 10-50 mM, or about 25 mM. The stabilizing agent may be present in a concentration of about 1-500 mM, for example, 10-50 mM, or about 25 mM. The buffering agent may be present in a concentration of about 1-500 mM, for example, 5-200 mM or about 75 mM. In one embodiment the sodium gluconate may be present in a concentration of about 25 mM, the sodium silicate may be present in a concentration of about 25 mM, and the sodium phosphate may be present in a concentration of about 75 mM.

The sample can be any biological sample that contains or that is suspected of containing DNA, for example, chromosomal DNA or extrachromosomal DNA, including mitochondrial DNA. The sample can be of prokaryotic or more typically of eukaryotic origin. The sample may contain hair, for example, human hair. The method is effective for other samples, including teeth, bone. The sample does not need to be ground or otherwise mechanically disrupted prior to extraction, although such disruption or grinding may be used if desired.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1. Comparison of the method of the invention to the method of Wilson et al. for extraction of mitochondrial DNA from hair shafts.

FIG. 2. Gel electrophoresis results of the amplification of mitochondrial DNA sequences extracted from human, hog, cat and horse hair containing no roots.

FIG. 3. Genotyping results from mitochondrial DNA extracted from different samples by different methods. Sample 1 is a head hair shaft extracted by the method of the invention. Sample 2 is a pubic hair shaft extracted by the method of the invention. Sample 3 is a public hair shaft extracted by the method of Wilson et. al. Sample 4 is a 2 mm punch of blood-spotted FTA paper extracted by the method of the invention. Sample 5 is 50 μl of whole blood extracted with the Qiamp blood mini kit (Qiagen Corporation).

### DETAILED DESCRIPTION OF THE INVENTION

The present invention provides compositions, methods, and kits for use in purifying DNA from a biological sample. The invention provides novel lysis reagents that extract DNA from biological samples with high efficiency without the need for toxic organic chemicals. The invention also provides methods and kits for purifying DNA that include the novel lysis reagents.

The Biological Sample

The biological sample can be any material composed of or containing any material of biological origin. Samples may include but are not limited to human and animal tissues, blood, bone, skin, hair, plant leaves, seeds, shootsi and stalks, bacterial and cell cultures, formalin-fixed tissue, blood spotted on preservative papers or solid matrices (such as FTA paper from Whatman), and the like. The sample need not contain only the biological material. The sample may also consist of a biological material on or in a physical matrix such as a stain of a bodily fluid on a piece of fabric, a piece of tissue embedded in a piece of wood, or a scraping of dirt containing a hair fragment.

Lysis Reagent

Surprisingly, the applicants have found that a highly basic solution containing an effective concentration of a chelating agent and a stabilizing agent, optionally containing a buffering agent, is highly effective at extracting DNA from tissue and other nucleic acid-containing samples. In particular, the applicants have found that solutions containing an effective concentration of a chelating agent such as sodium gluconate, an effective concentration of a stabilizing agent such as sodium silicate, and an effective concentration of a buffering agent such as sodium phosphate, are highly effective for DNA extraction.

The lysis reagent is strongly basic by virtue of the presence of a relatively high concentration of a base such as an alkali metal hydroxide, an alkaline earth metal hydroxide, or ammonium hydroxide. The skilled artisan will recognize that other strong bases may be used in place of these bases. The base typically is present at a final concentration of 0.1-5 M/L, advantageously 1-5M, and advantageously at a pH that is higher than about 12. In a typical solution according to the invention, the base is 1.5-2M, advantageously about 1.8M sodium hydroxide. In the context of the present invention, a highly basic solution is a solution having a pH of at least 12, and advantageously having a pH of at least about 13.

The chelating agent may be any composition that is effective for chelating metal ions. Examples of chelating agents are well known in the art and include sodium gluconate and EDTA although the skilled artisan will recognize that other chelating agents may be used, alone or in combination. The chelating agent can be present at a concentration of 1-500 mM, typically 1-100 mM, and advantageously about 25 mM.

The stabilizing agent is a reagent that stabilizes the solution and that applicants believe, without being bound by theory, stabilizes the DNA that is extracted from the source tissue or material. The stabilizing agent can be, for example, sodium metasilicate, sodium silicate, sodium sesquisilicate, sodium aluminosilicate, sodium fluorosilicate, and can be present at a concentration of about 1 mM to about 500 mM, typically 1-100 mM, and advantageously about 25 mM.

The buffering agent that optionally is present may be any agent that can act as a pH buffer. Such agents are well known in the art and include tetrasodium pyrophosphate, sodium citrate, sodium carbonate, trisodium nitrilotriacetic acid, sodium fluoroborate, sodium borate, and sodium triphosphate. The buffering agent can be present at a concentration of 0-500 mM, typically 1-200 mM, and advantageously at about 25-150 mM.

In a particularly advantageous embodiment of the invention, the base is present at a concentration of about 1.5-2M, the chelating agent is present at a concentration of about 10-50 mM, the stabilizing agent is present at a concentration of about 15-50 mM, and the buffering agent is present at a concentration of about 50-100 mM. In a particularly advantageous embodiment, the solution comprises 1.8M sodium hydroxide, 25 mM sodium gluconate, 25 mM sodium silicate and 75 mM sodium phosphate.

Without being bound by any theory, applicants believe that the enhanced properties of the lysis reagents of the invention may be due in at least some cases to the chelating properties of the reagent.

The skilled artisan will recognize that the solution components may be combined in different combinations and at different concentrations to achieve optimal extraction of DNA from nearly any biological sample. The skilled artisan also will recognize that the sodium salts described above for these additives could be replaced by salts containing other suitable counterions, for example, potassium or lithium.

The invention also provides novel methods for extracting and purifying DNA that employ the Lysis Reagents described above.

Binding Reagent

For the extraction of DNA, the binding reagent is a solution comprised of a chaotropic agent such as guanidine hydrochloride, and a concentrated buffer system such as a mixture of acetic acid and potassium acetate. The buffers in the binding reagent neutralize the Lysis Reagent and are formulated so that when the Binding Reagent is Mixed with the Lysis Reagent, the pH of the resulting mixture will allow the binding of the DNA to the Binding Matrix. The pH range of the mixture of Lysis Reagent and Binding Reagent is typically 4.0-7.5. Optionally, the binding reagent may contain an alcohol such as ethanol, isopropanol, at a concentration of 2-80%. In one embodiment, the Binding Reagent is comprised of 4.2 M, or about 4.2 M, guanidine hydrochloride, 0.9 M or about 0.9 M, potassium acetate, and 0.6 M or about 0.6 M acetic acid, pH 4.4.

Wash Solution

The Wash Solution typically comprises a low concentration of a chaotropic agent and an alcohol such as ethanol or isopropanol. For example, the wash solution may comprise 1M guanidine hydrochloride and 60% ethanol, pH 7.0.

Binding Device and Binding Matrix

The Binding Device is any device containing a Binding Matrix that selectively binds DNA over proteins and other biochemical components of biological samples. Such devices are commercially available from a wide range of companies (Qiagen, Marligen, Clontech, Promega, Genomed) in a wide variety of formats including single tubes, 96-well plates, 384-well plates. The Binding Matrix may be a membrane, gel, or particles or any other material that is commonly used for separation of biological molecules including, for example, silica membranes, silica resin, glass milk, and ion-exchange resins. The Binding Matrix may be part of the binding device or may be added separately. Membranes are usually incorporated into the device while loose resin is usually supplied separately and then added to a separation device. The devices may be constructed in any format desired. A typical single tube device contains a non-binding porous frit on the bottom, one or more layers of silica membrane on top, and finally a retaining ring to ensure that the frit and membrane stay seated at the bottom of the tube. Such devices are convenient in that after each liquid addition, the added liquid may be drawn through the membrane by centrifugation or vacuum. The Binding Device may also be a plate composed of many individual wells. Plates comprised of 96, 384, and 1536 wells are commonly used for high-throughput parallel analysis. Plate formats are constructed to use centrifugation or vacuum, or both, to draw liquid through the Binding Matrix.

Elution Buffer

If the Binding Matrix is silica-based, then the Elution Buffer is typically water or TE buffer (10 mM Tris, 1 mM EDTA, pH 8.0), but may be any solution of low ionic strength of pH between 7.0 and 8.5. If the Binding Matrix is an ion-exchange matrix, then the elution buffer is typically a high salt solution, such as 1.25 M sodium chloride, 50 mM Tris, pH 7.5. The exact composition of the elution buffer is not critical as long as it results in the elution of the DNA from the Binding Matrix.

General Procedure

The sample is added to a fixed volume of Lysis Reagent. The sample is agitated so that all of the sample comes into contact with the lysis reagent. The sample in lysis reagent is then incubated for 0.5-60 minutes at 20° C.-99° C., the temperature and time being dependent upon the nature of the sample. Simple cultured cells and fresh tissue may be incubated for 1-2 minutes at room temperature (20-25° C.) while hair, formalin-fixed tissue and plant material may need to be incubated for 10 minutes at 65° C. or higher. Following incubation, 1-50 volumes of Binding Reagent is added to the Lysis Reagent containing the sample and the solution is mixed thoroughly. For DNA extraction, the Binding Solution neutralizes the base when combined with the Lysis Solution, and provides the optimum pH and salt conditions for binding of the DNA to the membrane in the Binding Device. The neutralized solution is added to the Binding Device and the liquid is drawn through the silica membrane by gravity, centrifugation, vacuum, or pumping. The solution containing the sample is then added to the binding device to allow the nucleic acid to bind to the nucleic acid Binding Matrix. Once the solution is added, and the nucleic acid has bound to the matrix, the excess liquid containing the non-binding components of the sample is removed by centrifugation, vacuum, gravity, pumping, or any other method know in the art for separating bound from unbound components. Wash Solution is added to the Binding Device and is drawn through or across the Binding Matrix to wash out residual chaotropic salts, buffer salts and residual components that may be binding with low affinity to the binding matrix. The DNA which binds to the matrix with high affinity, remains bound to the matrix. The purpose of the wash step is to wash away proteins, biochemical, and other components of the sample while retaining the DNA on the silica matrix. Additional washes with solutions known in the art may be performed at this point to remove specific contaminants that would otherwise co-purify with the DNA. Examples of such contaminants are RNA, endotoxins, plant resins, and polyphenolic compounds. Elution Buffer is added in a small volume to the Binding Matrix. The Elution Buffer may be allowed to incubate on the Binding Matrix for 0-60 minutes to allow the nucleic acid to be freed from the matrix and go into the solution phase. After this incubation, the elution buffer, containing the nucleic acid, is recovered from the Binding Matrix by centrifugation, vacuum, pumping, or any other method known in the art.

The procedure given above is not meant to be limiting as many variations are possible as may be made by those skilled in the art of purification of RNA and DNA. The present invention, thus generally described, will be understood more readily by reference to the following examples, which are provided by way of illustration and are not intended to be limiting of the present invention.

### EXAMPLE 1

Purification of Mitochondrial DNA from Human, Dog, Cat and Horse Hair Containing No Root.

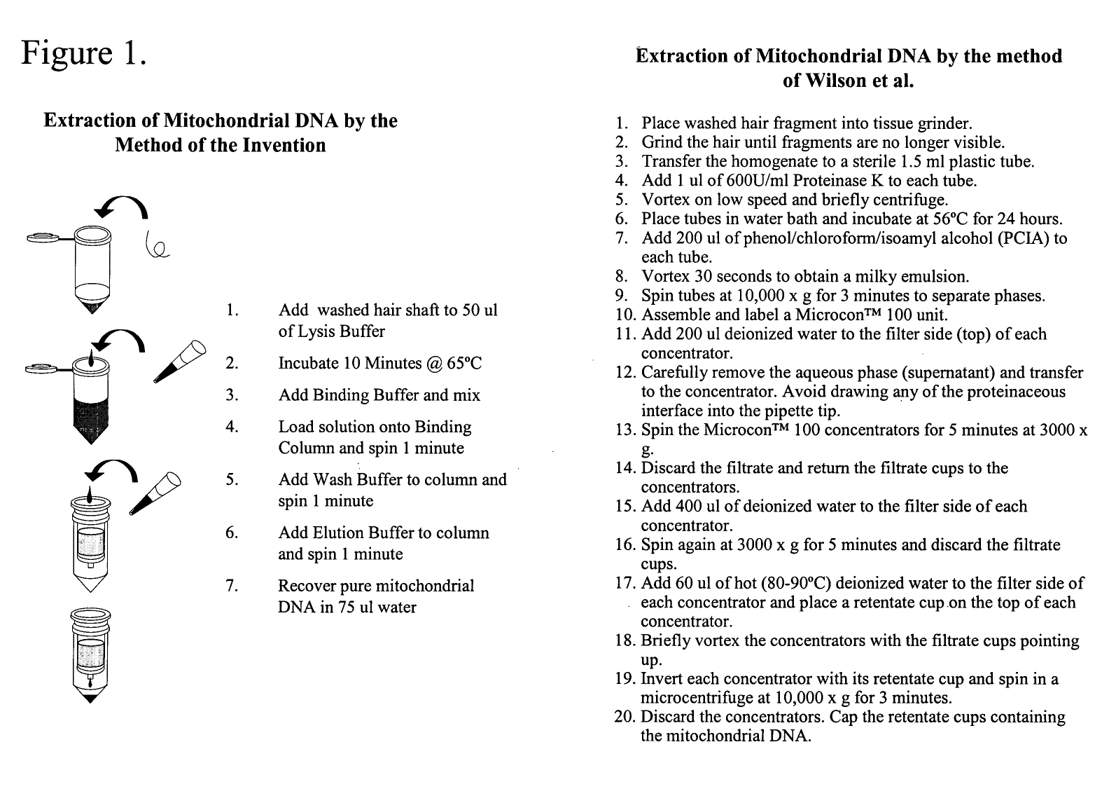

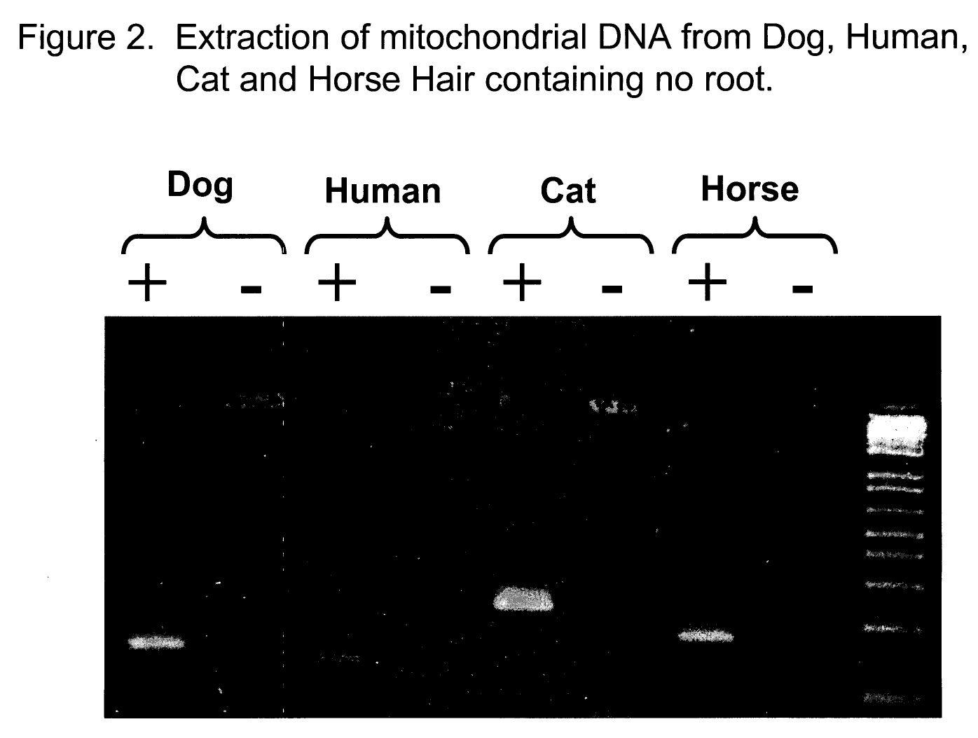

A 2 cm hair shaft from each species containing no hair root was added to a 2 ml microcentrifuge tube. One milliliter of 5% Terg-a-zyme in deionized water was added to the hair shaft and incubated in a sonicating water bath at room temperature for 20 minutes. The Terg-a-zyme wash solution was discarded and 1 ml of 100% ethanol was added to the hair shaft, covering it entirely. The hair shaft was soaked for 5 minutes in the ethanol and then as much ethanol as possible was removed using a disposable pipette. Fifty microliters of Lysis Reagent (1.8 M NaOH, 25 mM sodium gluconate, 25 mM sodium silicate, 75 mM sodium phosphate) was added to each tube containing a hair shaft and a pipette tip was used to push the hair shaft into the liquid at the bottom of the tube. The hair shaft was incubated in Lysis Buffer for 10 minutes at 60° C. and the tubes were vortexed briefly after five minutes and at the end of the 10 minute incubation to facilitate physical disruption of the hair. Six hundred milliliters of Binding Buffer containing 4.2 M guanidine hydrochloride, 0.6 M acetic acid, and 0.9 M potassium acetate, pH 4.4 was added to the digested hair sample and mixed thoroughly by vortexing. Each sample was added to a DNA Binding Column sitting in a receiver tube containing a silica membrane and centrifuged for 1 minute at 12,000×g. The column flow through was discarded and the column was replaced in the receiver tube. Seven hundred microliters of Wash Solution comprising 1M guanidine and 60% ethanol was added to the column and incubated for 2 minutes at room temperature. The column was then centrifuged for 1 minute at 12,000×g. The receiver tube was discarded and the DNA Binding Column was placed in a clean receiver tube. Elution buffer comprising 10 mM Tris, 1 mM EDTA, pH 8.0 was heated to 65° C. and 75 microliters of the heated Elution Buffer was applied to the center of the silica membrane and incubated for 1 minute. The column was centrifuged for 1 minute at 12,000×g and the flow-through containing purified mitochondrial DNA was collected in the receiver tube and stored at −20° C. A comparison of the method of the invention to the procedure of Wilson et al for purification of mitochondrial DNA from hair shafts is shown in FIG. 1. The purified mitochondrial DNA was amplified by the polymerase chain reaction using the following primer sequences: human, forward primer—CCCCATGCTTACAAGCAAGT; human, reverse primer—TGGCTTTATGTACTATGTAC; dog, forward primer—GAACTAGGTCAGCCCGGTACTT, dog, reverse primer—CGGAGCACCAATTATTAACGGC; cat, forward primer—TTCTCAGGATATACCCTTGACA; cat, reverse primer—GAAAGAGCCCATTGAGGAAATC and horse, forward primer—CCCTAAGCCTCCTAATCCGT; horse, reverse primer—AGGAATGATGGGGCAAGTAA. PCR reaction mixes contained 20 mM Tris-HCl (pH 8.4), 1.5 mM MgCl2, 200 uM each dNTP, 200 nM each primer, and 1 unit of Platinum Taq Polymerase (Invitrogen Corporation). The template mitochondrial DNA was denatured for 10 minutes at 95° C., and then amplified with 35 cycles of 94° C. denaturation for 30 seconds, 55° C. primer annealing for 30 seconds, and 72° C. primer annealing for 1 minute. At the completion of the 35 cycles, the reactions were extended at 72° C. for an additional 1 minute. PCR reactions were separated by agarose gel electrophoresis and DNA bands were visualized by ethidium bromide staining. Bands of the expected size were detected for each of the hair shafts that were processed, and no staining was evident in the negative controls. (see FIG. 2.)

### EXAMPLE 2

Genotypint Mitochondrial DNA

Five different samples containing DNA were obtained from the same person. Sample 1 was a 2 cm piece of hair shaft obtained from the head with no root attached. Samples 2 and 3 were 2 cm shafts of pubic hairs without no root attached. Sample 4 was a 2 cm punch of FTA paper that had been spotted with whole blood. Sample 5 was 50 μl of whole blood. Mitochondrial DNA was extracted from samples 1 and 2 by the method of the invention as described in Example 1. DNA was extracted from sample 3 by the method of Wilson et al. For extraction of mitochondrial DNA by the method of Wilson et. al., micro tissue grinders were used to grind hairs for DNA extraction. These grinders consist of matched sets of mortars and pestles. To prevent contamination from exogenous DNA, the grinders were carefully rinsed with deionized water and were scrubbed with 5% Terg-a-zyme™ using cotton tip applicators. The grinders were then rinses with deionized water and were soaked for 20 minutes in 200 microliters of 1 normal sulfuric acid. The grinders were rinsed thoroughly with deionized water and then spun at 10,000×g in a microcentrifuge to remove all remaining traces of liquid.

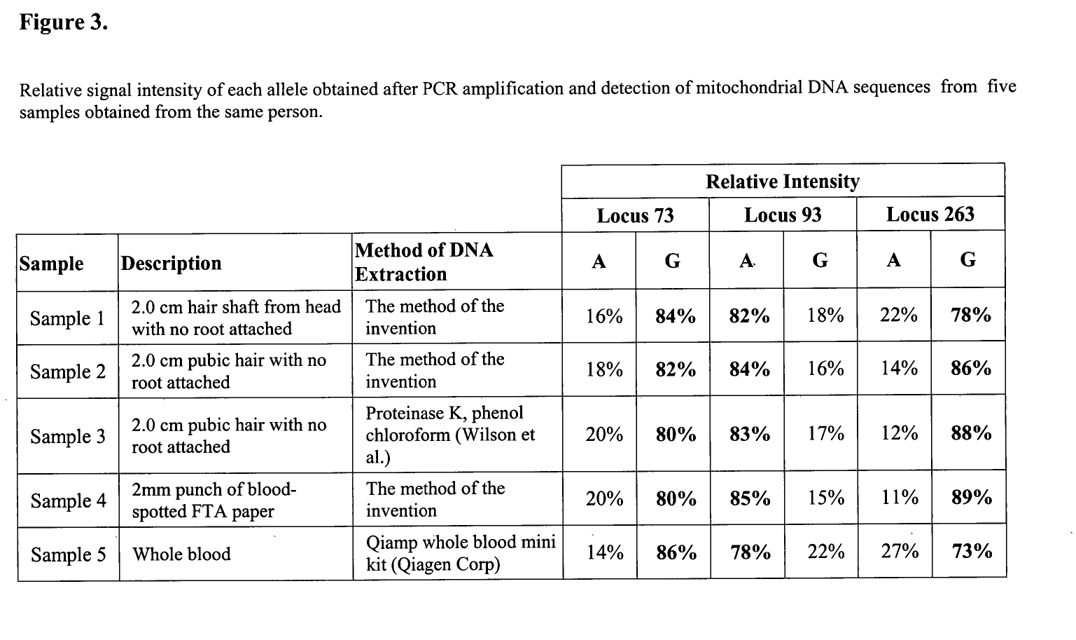

A 2 cm hair shaft was added to a 2 ml microcentrifuge tube. One milliliter of 5% Terg-a-zyme in deionized water was added to the hair shaft and incubated in a sonicating water bath at room temperature for 20 minutes. The Terg-a-zyme wash solution was discarded and 1 ml of 100% ethanol was added to the hair shaft, covering it entirely. The hair shaft was soaked for 5 minutes in the ethanol and then as much ethanol as possible was removed using a disposable pipette. To the tube containing the micro tissue grinder was added 200 uL of stain extraction buffer followed by the 2 cm hair shaft. The pestle was moved up and down to force the hair into the bottom of the mortar. The hair was then ground until no fragments were visible. The pestle was removed from the mortar and the homogenate liquid was transferred to a sterile 1.5 ml microcentrifuge tube. One microliter of 600 U/mL proteinase K was added to the tube and mixed thoroughly by vortexing at low speed. The tube was centrifuged briefly to bring all of the liquid to the bottom of the tube and the tube was then incubated at 56° C. for 24 hours. After incubation, 200 microliters of phenol/chloroform/isoamyl alcohol (PCIA, 25:24:1) was added to the tube and then vortexed for 30 seconds to prepare a milky emulsion. The tube was then centrifuged for 3 minutes at 12,000×g to separate the aqueous and organic phases. A Microconrm 100 microconcentrator (Millipore Corporation, Billerica, Mass.) was assembled, labeled, and e prepared for use by adding 200 microliters of deionized water on the filter side (top) of each concentrator. The aqueous phase (supernatant of approximately 200 microliters)of the phenol-chloroform-isoamyl alcohol was carefully removed from the tube and transferred to the microconcentrator taking special care to avoid drawing any of the proteinaceous interface into the pipette tip. The Microcon™ 100 microconcentrator was centrifuged for 5 minutes at 3000×g. The filtrate was discarded and the filtrate cup was returned to the concentrator. Four hundred microliters of deionized water was added to the retentate side of the microconcentrator and the microconcentrator was centrifuged at 3000×g for 5 minutes and the filtrate was discarded. Sixty microliters of deionized water at 80° C. was added to the retentate side of the Microcon™ 100 concentrator and a retentate cup was placed on the top of each concentrator. The microconcentrator was inverted with its retentate cup and centrifuged for 10,000×g for 3 minutes. The retentate cup contains the solution containing the mitochondrial DNA. DNA was extracted from sample 4 (blood spotted onto FTA paper) by the method of the invention. For extraction of mitochondrial DNA from dried blood stored on FTA paper (Whatman), a 2 mm circle of spotted blood was punched from the dried blood spot on the FTA paper and was added to a 1.5 ml microcentrifuge tube. One hundred microliters of Lysis Reagent (1.8 M NaOH, 25 mM sodium gluconate, 25 mM sodium silicate, 75 mM sodium phosphate) was added to the tube containing the hair shaft and the tube was vortexed vigorously for 1 minutes. The solution was incubated for 10 minutes at 60° C. and then vortexed again for 1 minute. Six hundred milliliters of Binding Buffer containing 4.2 M guanidine hydrochloride, 0.6 M acetic acid, and 0.9 M potassium acetate was added to the digested hair sample and mixed thoroughly by vortexing. The entire sample was added to a DNA Binding Column sitting in a receiver tube containing a silica membrane and centrifuged for 1 minute at 12,000×g. The column flow through was discarded and the column was replaced in the receiver tube. Seven hundred microliters of Wash Solution comprising 1M guanidine and 60% ethanol was added to the column and incubated for 2 minutes at room temperature. The column was then centrifuged for 1 minute at 12,000×g. The receiver tube was discarded and the DNA Binding Column was placed in a clean receiver tube. Elution buffer comprising 10 mM Tris, 1 mM EDTA, pH 8.0 was heated to 65° C. and 75 microliters of the heated Elution Buffer was applied to the center of the silica membrane and incubated for 1 minute. The column was centrifuged for 1 minute at 12,000×g and the flow-through containing purified mitochondrial DNA was collected in the receiver tube and stored at −20° C. DNA from sample 5 was extracted from whole blood with the Qiamp DNA Blood Mini Kit (Qiagen Corporation) according to the instructions supplied with the kit. Ten microliters of each purified DNA sample was then amplified and genotyped using the reagents and protocol supplied in the Marligen Mitochondrial DNA Screening System (Marligen Biosciences, Ijamsville, Md.). Polymerase chain reaction amplification (PCR) was performed using primers specific for mitochondrial DNA hypervariable region II. The PCR products were genotyped by allele-specific hybridization using a suspension array of allele-specific oligonucleotides immobilized on latex beads according to the manufacturers instructions. Results for each allele are expressed as a percentage of the total signal for all possible alleles at each locus (FIG. 3). The results obtained demonstrate that hair samples extracted with the method of the invention give identical results to those obtained with the method of Wilson et al ( proteinase K with phenol-chloroform-isoamyl alcohol extraction) and identical results to those obtained with DNA extracted from whole blood using a commercial kit. Similarly, DNA extracted from blood spotted on FTA paper gave identical results to those obtained with DNA extracted from whole blood using a commercial kit.

References

1. Baker et al. (2001), Journal of Forensic Sciences 46:126-130.

2. Bookjans et al. (1984), Anal Biochem 141: 244-247.

3. Charity et al, Procedural Improvements in Mitochondrial DNA Analysis: Validation and Implementation of a New Hair Extraction Procedure and a Modified Amplification/Sequencing Primer. The Bode Technology Group Inc., 7364 Steel Mill Drive, Springfield, Va., 22150. Abstract presented at the 9^{th} Promega Meeting.

4. Chomczynski and Sacchi (1987) Anal Biochem. 162:156-9.

5. Crouzillat et al. (1987) Theor Appl Genet. 74: 773-780.

6. Herrmann (1982). In: Edelman M, Hallick R B and Chua N H (eds), Methods in Chloroplast Molecular Biology, pp 259-280. Elsevier Biomedical Press, Amsterdam.

7. Mackenzie (1994) Plant Mol Biol Manual D3, pp 1-12. Kluwer Academic Publishers, Dordrecht.

8. Triboush et al, Plant Molecular Biology Reporter 16: 183-189, 1998.

9. Wilson and Chourey (1984) Plant Cell Rep 3: 237-239.

10. Wilson et al. Biotechniques. 1995. April;18(4):662-9.

What is claimed is:

1. A method for extracting DNA from a biological sample, comprising contacting the sample with a highly basic solution comprising an effective concentration of a chelating agent, an effective concentration of a stabilizing agent and an effective concentration of a buffering agent.

2. The method according to claim 1, wherein said chelating agent is an alkali metal gluconate salt.

3. The method according to claim 1, wherein said stabilizing agent is an alkali metal silicate salt.

4. The method according to claim 1, wherein said buffering agent is an alkali metal phosphate salt.

5. The method according to claim 1, wherein said chelating agent is sodium gluconate, said stabilizing agent is sodium silicate, and said buffering agent is sodium phosphate.

6. The method according to claim 2, wherein said chelating agent is present in a concentration of about 1-500 mM.

7. The method according to claim 3, wherein said stabilizing agent is present in a concentration of about 1-500 mM.

8. The method according to claim 4, wherein said buffering agent is present in a concentration of about 1-500 mM.

9. The method according to claim 6, wherein said chelating agent is present in a concentration of about 10-50 mM.

10. The method according to claim 7, wherein said stabilizing agent is present in a concentration of about 10-50 mM.

11. The method according to claim 8, wherein said buffering agent is present in a concentration of about 5-200 mM.

12. The method according to claim 5, wherein said sodium gluconate is present in a concentration of about 25 mM, said sodium silicate is present in a concentration of about 25 mM, and said sodium phosphate is present in a concentration of about 75 mM.

13. The method according to claim 1, wherein said sample comprises hair.

14. The method according to claim 13, wherein said hair is a human hair.

15. The method according to claim 13, wherein said hair is not ground prior to extraction.

16. The method according to claim 1, wherein the sample comprises a biological sample on or within a solid matrix.

17. The method according to claim 16, wherein the solid matrix is paper.

18. The method according to claim 17, wherein the paper is FTA® paper.

19. The method according to claim 16, wherein the sample comprises blood.

20. The method according to claim 17, wherein the sample comprises blood and the solid matrix comprises FTA® paper.
